(19) United States
(12) Patent Application Publication
(10) Pub. No.: US 2011/0046636 A1
(43) Pub. Date: Feb. 24, 2011

(54) Surgical Guide Instrument Capable of Omni-Directional Positioning and Omni-Directional Positioning Unit Thereof

(75) Inventors: Ming-Chang Wu, Guishan Shiang (TW); Hong-Yu Zhu, Guishan Shiang (TW); Hung-Sheng Tien, Guishan Shiang (TW); Kuo-Tung Kao, Guishan Shiang (TW); Chi-Bin Wu, Guishan Shiang (TW)

Correspondence Address: STITES & HARRISON PLLC, Suite 900, 1199 N. Fairfax Street, Alexandria, VA, 22314, US

(73) Assignee: AccuMIS Inc., Zhonghe City (TW)

(21) Appl. No.: 12/619,867

(22) Filed: Nov. 17, 2009

(30) Foreign Application Priority Data
Aug. 21, 2009 (TW) 098128113

(51) Int. Cl. A61B 19/00

(52) U.S. Cl. 606/130

(57) ABSTRACT

A surgical guide instrument capable of omni-directional positioning and an omni-directional positioning unit thereof are provided. The surgical guide instrument includes the omni-directional positioning unit, which is coupled with the surgical instrument. The omni-directional positioning unit is configured as a pyramid, and a plurality of reflectors is deposited on vertices of the pyramid, respectively. As the reflectors of the omni-directional positioning unit are arranged as a three-dimensional pyramid, the omni-directional positioning unit can be connected in any position to the surgical instrument and be detected correctly by a surgical guide system, thereby reducing surgical complexity.

### BACKGROUND OF THE INVENTION

1. Technical Field

The present invention relates to a surgical guide instrument capable of omni-directional positioning and an omni-directional positioning unit thereof. More particularly, the present invention relates to a surgical guide instrument capable of omni-directional positioning and applicable to a surgical guide system, and an omni-directional positioning unit of the surgical guide instrument.

2. Description of Related Art

Prior to performing orthopedic surgical procedures on a patient, it is common practice to take images of the patient's lesion with a proper medical imaging instrument, such as one based on tomography or magnetic resonance imaging, so as to assist the surgeon in determining the location and size of the lesion and planning the incision point, direction, and depth of the operation accordingly. As the location of a lesion is mostly determined on the basis of the surgeon's anatomical knowledge and clinical experience, precise preoperative planning is often unattainable. Therefore, in case of a complex lesion, a surgeon may have to look for the lesion while cutting through the tissues.

FIG. 1 illustrates a conventional surgical guide system 100 while FIG. 2A and FIG. 2B show two different structures of a conventional positioning device 102.

Referring to FIG. 1, the surgical guide system 100 serves to increase the precision of preoperative planning and is configured for providing sufficient spatial information and thus enabling a surgeon to plan the paths of surgical instruments during an operation. The surgical guide system 100 includes an image-capturing device 101, a positioning device 102, and an image analysis device 103. With the patient lying on the operation table, the image-capturing device 101 takes real-time images of the patient's lesion and sends the images to the image analysis device 103 for analysis.

The positioning device 102, which is installed on a surgical instrument 104, can be an optical positioning device. In this case, optical signals sent by the positioning device 102 are continuously monitored by a tracking device 105 of the image analysis device 103 so as to obtain spatial coordinates of the surgical instrument 104. The image analysis device 103 analyzes and calculates the spatial coordinates of the surgical instrument 104 with reference to the images of the lesion, thereby precisely determining the position of the surgical instrument 104 in relation to the lesion.

As shown in FIG. 2A and FIG. 2B, the conventional positioning device 102 includes three or four reflectors 106 configured for generating optical signals. However, as the reflectors 106 are arranged in the same plane, the tracking device 105 cannot detect all the reflectors 106 if any one of the reflectors 106 is obstructed due to an improper orientation. Lacking the spatial information of a certain reflector 106, the image analysis device 103 is unable to calculate the accurate position of the surgical instrument 104, and in consequence the precision with which to determine the position of the surgical instrument 104 is lowered significantly.

### BRIEF SUMMARY OF THE INVENTION

It is an objective of the present invention to provide a surgical guide instrument capable of omni-directional positioning and an omni-directional positioning unit thereof, wherein reflectors of the omni-directional positioning unit are deposited at vertices of a three-dimensional pyramid, respectively. Therefore, the reflectors are less likely to be obstructed by other objects or instruments than if the reflectors are arranged in the same plane. As a result, enhanced precision in positioning is achieved.

It is another objective of the present invention to provide a surgical guide instrument capable of omni-directional positioning and an omni-directional positioning unit thereof, wherein the omni-directional positioning unit increases the precision in positioning and thereby enhances the guiding accuracy of the surgical guide instrument. Consequently, surgical complexity is reduced.

To attain the above and other objectives, the present invention provides a surgical guide instrument capable of omni-directional positioning, wherein the surgical guide instrument includes a surgical instrument and an omni-directional positioning unit coupled with the surgical instrument. The omni-directional positioning unit is configured as a pyramid and includes a plurality of reflectors. The pyramid has a base and a plurality of faces extending from the base. The reflectors are deposited at vertices of the pyramid, respectively.

To attain the above and other objectives, the present invention provides an omni-directional positioning unit configured as a pyramid and including a plurality of reflectors. The pyramid has a base and a plurality of faces extending from the base while the reflectors are deposited at vertices of the pyramid, respectively.

Implementation of the present invention at least involves the following inventive steps:

1. The reflectors arranged as a pyramid are less likely to be obstructed, thereby increasing the precision in positioning.

2. With increased precision in positioning, the surgical instrument can be guided with enhanced accuracy to lower surgical complexity.

### BRIEF DESCRIPTION OF THE SEVERAL VIEWS OF THE DRAWINGS

A detailed description of further features and advantages of the present invention is given below so that a person skilled in the art can understand and implement the technical contents of the present invention and readily comprehend the objectives and advantages thereof by reviewing the teachings disclosed herein and the appended claims in conjunction with the accompanying drawings, in which:

FIG. 1 illustrates a conventional surgical guide system;

FIG. 2A shows the structure of a conventional positioning device;

FIG. 2B shows another structure of the conventional positioning device;

FIG. 3 is an exploded perspective view of an embodiment of a surgical guide instrument capable of omni-directional positioning according to the present invention;

FIG. 4 is an exploded perspective view of another embodiment of the surgical guide instrument capable of omni-directional positioning according to the present invention;

FIG. 5A is a perspective view of a first embodiment of an omni-directional positioning unit according to the present invention;

FIG. 5B is a perspective view of a second embodiment of the omni-directional positioning unit according to the present invention;

FIG. 6A is a perspective view of a third embodiment of the omni-directional positioning unit according to the present invention;

FIG. 6B is a perspective view of a fourth embodiment of the omni-directional positioning unit according to the present invention;

FIG. 7A is a perspective view of a fifth embodiment of the omni-directional positioning unit according to the present invention; and

FIG. 7B is a perspective view of a sixth embodiment of the omni-directional positioning unit according to the present invention.

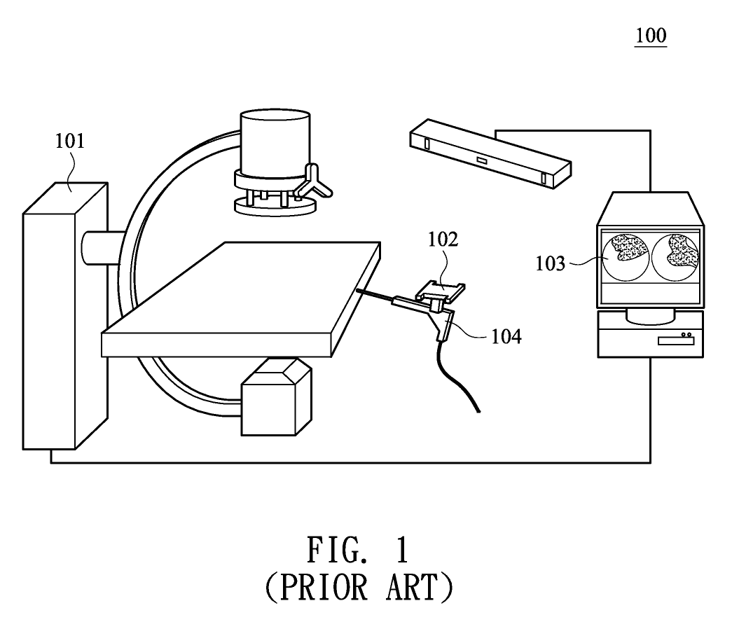

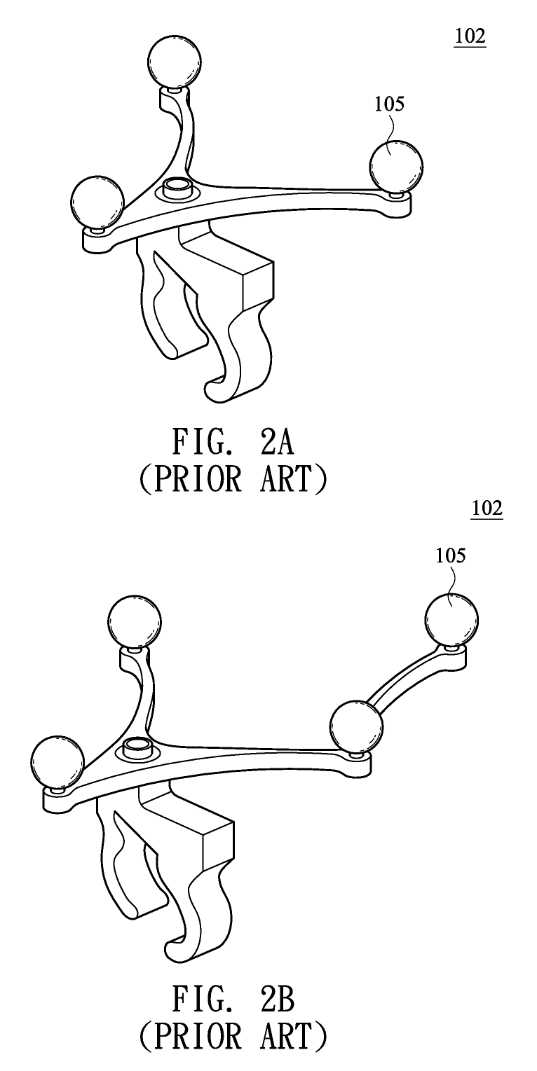

### DETAILED DESCRIPTION OF THE INVENTION

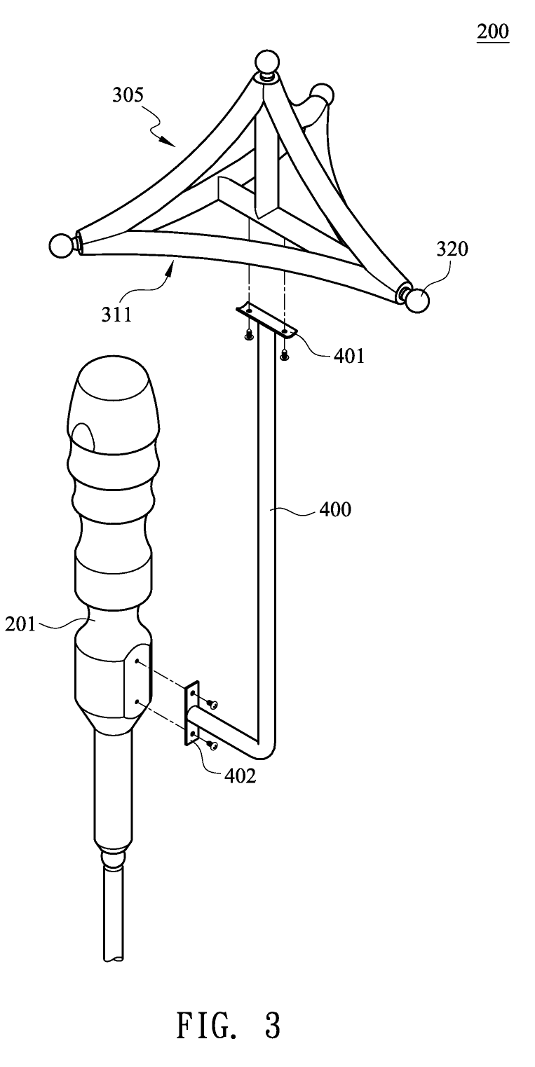

Referring to FIG. 3, according to an embodiment of the present invention, a surgical guide instrument 200 capable of omni-directional positioning includes a surgical instrument 201 and an omni-directional positioning unit 305.

As shown in FIG. 3, the surgical instrument 201 can be any surgical instrument for use in a surgical operation, such as a scalpel, a surgical clamp, and so on.

As shown in FIG. 3, the omni-directional positioning unit 305 is coupled with the surgical instrument 201. The omni-directional positioning unit 305 can be implemented in various ways, as discussed further below. Since the position of the omni-directional positioning unit 305 can be determined via detection from any direction, the omni-directional positioning unit 305 can be connected in any position to the surgical instrument 201 as appropriate.

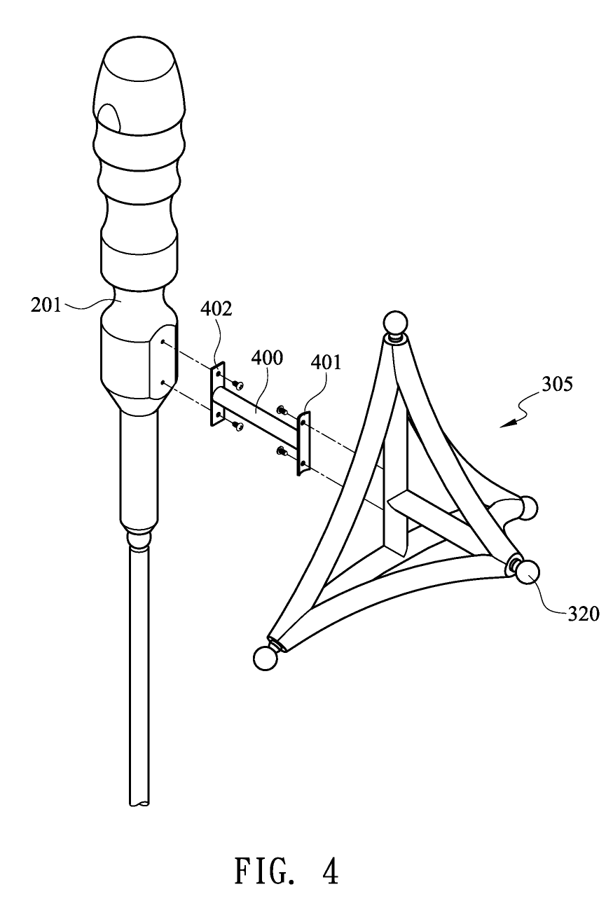

With reference to FIG. 3 and FIG. 4, the omni-directional positioning unit 305 includes a supporting element 400 having a first end portion 401 and a second end portion 402. The first end portion 401 is coupled with a base 311 of the omni-directional positioning unit 305 while the second end portion 402 is coupled with the surgical instrument 201. The configuration and length of the supporting element 400 may vary according to practical needs. For instance, the supporting element 400 may have a bent portion (as shown in FIG. 3), or the supporting element 400 may have a relatively short length (as shown in FIG. 4), thus allowing the omni-directional positioning unit 305 to be fixed at an appropriate position for positioning the surgical instrument 201.

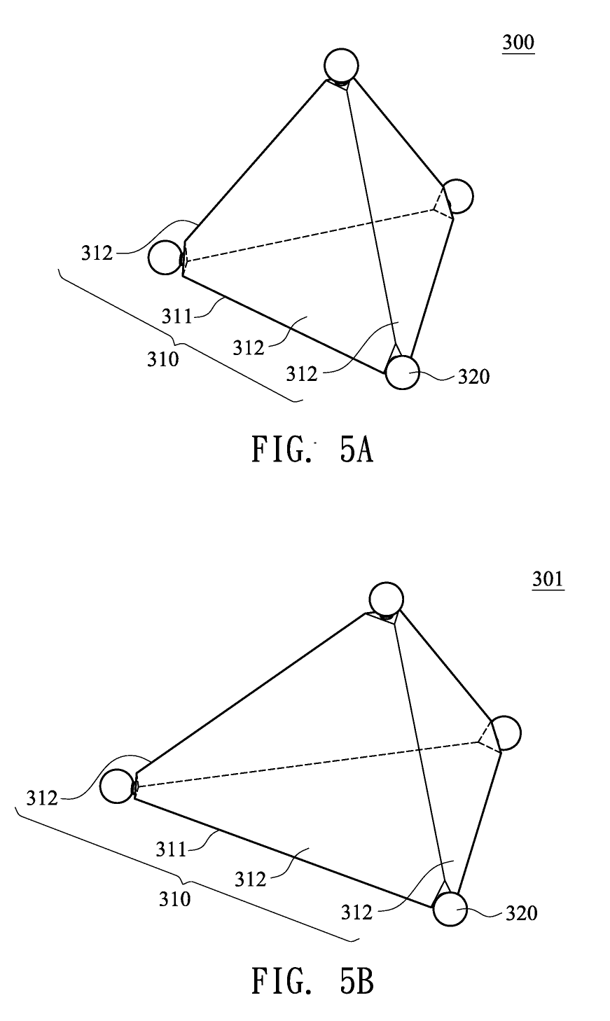

Referring to FIG. 5A, an omni-directional positioning unit 300 is configured as a pyramid 310 and includes a plurality of reflectors 320. As shown in FIG. 5A, the pyramid 310 has a base 311 and a plurality of faces 312, wherein each face 312 extends from the base 311 of the pyramid 310. In addition, the faces 312 and the base 311 jointly form four vertices.

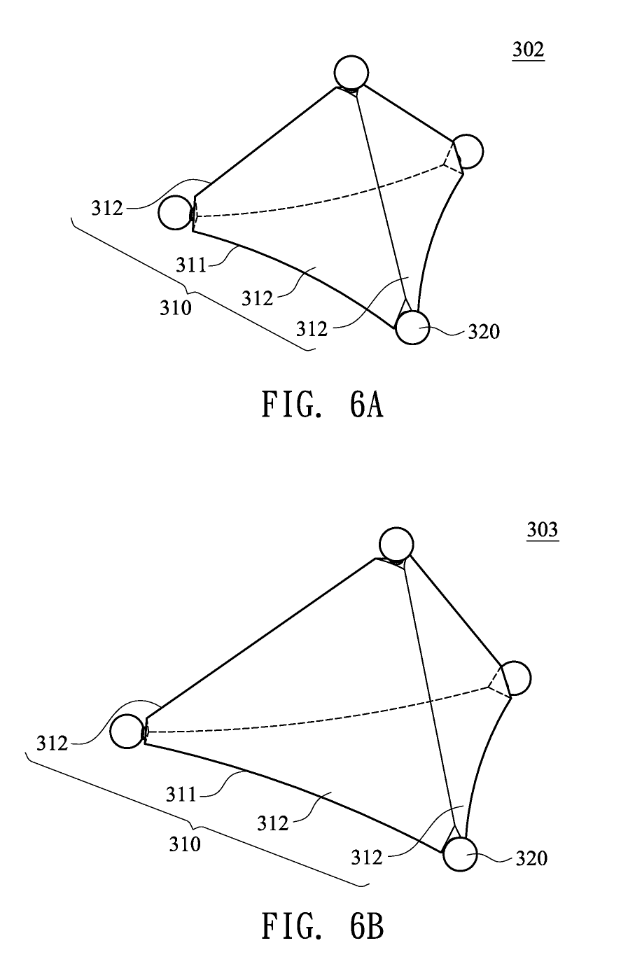

As shown in FIG. 5A, each reflector 320 is deposited at a corresponding one of the vertices of the pyramid 310. Therefore, the reflectors 320 of the omni-directional positioning unit 300 are arranged three-dimensionally. Furthermore, referring to FIGS. 5A, 5B, 6A, and 6B, the base 311, as well as the faces 312, of each of the omni-directional positioning units 300, 301, 302, 303 is a plate, wherein the plate is either a planar plate (as shown in FIG. 5A and FIG. 5B) or a plate having a curved surface (as shown in FIG. 6A and FIG. 6B).

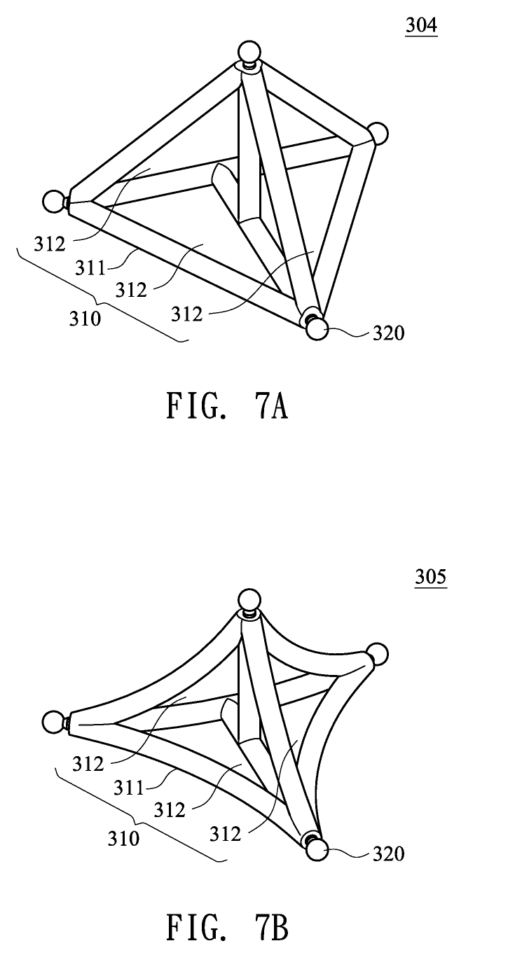

Referring to FIG. 7A and FIG. 7B, the pyramid 310 formed by each of the omni-directional positioning units 304, 305 includes a plurality of bars which connect the corresponding vertices of the pyramid 310, respectively. The bars may also be curved, as shown in FIG. 7B.

The pyramid 310 formed by each of the omni-directional positioning unit 300, 301, 302, 303, 304, 305 may vary in configuration according to practical needs. For example, the base 311 of the pyramid 310 is configured as an equilateral triangle while the faces 312 are configured also as equilateral triangles (as shown in FIG. 5A, FIG. 7A, and FIG. 7B) or as arbitrary triangles (as shown in FIG. 6A). Alternatively, referring to FIG. 5B and FIG. 6B, the base 311 of the pyramid 310 is configured as an arbitrary triangle while the faces 312 extending from the base 311 are configured also as arbitrary triangles.

As a surgical guide system uses an optical positioning device to determine the position of the surgical instrument 201, it is required that the reflectors 320 of each of the omni-directional positioning units 300, 301, 302, 303, 304, 305 emit optical signals so as for the surgical guide system to determine the position of the surgical instrument 201 accordingly. Therefore, each reflector 320 can be a reflective ball or an infrared reflector for reflecting the optical signals to the optical positioning device at any time, thus enabling real-time positioning of the surgical instrument 201.

Moreover, as the reflectors 320 are deposited at the vertices of the pyramid 310, respectively, the reflectors 320 are arranged in a three-dimensional manner. With the reflectors 320 being configured as a pyramid, the optical positioning device can detect the optical signals reflected by the reflectors 320 at whichever angle the reflectors 320 are placed beside the surgical instrument 201 or in whichever direction the optical positioning device makes the detection. Thus, the surgical instrument 201 can be detected in any position regardless of the placement angle of the reflectors 320, thereby allowing the surgical instrument 201 to be accurately positioned as well as lowering the complexity of surgical operation.

The foregoing embodiments are illustrative of the characteristics of the present invention so as to enable a person skilled in the art to gain insight into the contents disclosed herein and implement the present invention accordingly. The embodiments, however, are not intended to restrict the scope of the present invention. Hence, all equivalent modifications and variations made in the foregoing preferred embodiments without departing from the spirit and principle of the present invention should fall within the scope of the appended claims.

What is claimed is:

1. A surgical guide instrument capable of omni-directional positioning, comprising:
a surgical instrument; and
an omni-directional positioning unit coupled with the surgical instrument and characterized by:
being configured as a pyramid having a base and a plurality of faces extending from the base; and
comprising a plurality of reflectors each deposited at a corresponding one of vertices of the pyramid.

2. The surgical guide instrument of claim 1, wherein the omni-directional positioning unit further comprises a supporting element having a first end portion coupled with the base and a second end portion coupled with the surgical instrument.

3. The surgical guide instrument of claim 1, wherein the base is a plate.

4. The surgical guide instrument of claim 3, wherein each said face is a plate.

5. The surgical guide instrument of claim 1, wherein the pyramid comprises a plurality of bars connecting corresponding ones of the vertices, respectively.

6. The surgical guide instrument of claim 1, wherein the base is configured as an equilateral triangle.

7. The surgical guide instrument of claim 6, wherein each said face is configured as an equilateral triangle or an arbitrary triangle.

8. The surgical guide instrument of claim 1, wherein the base is configured as an arbitrary triangle.

9. The surgical guide instrument of claim 8, wherein each said face is configured as an arbitrary triangle.

10. The surgical guide instrument of claim 1, wherein each said reflector is a reflective ball or an infrared reflector.

11. An omni-directional positioning unit, characterized by:
being configured as a pyramid having a base and a plurality of faces extending from the base; and
comprising a plurality of reflectors each deposited at a corresponding one of vertices of the pyramid.

12. The omni-directional positioning unit of claim 11, further comprising a supporting element coupled with the base.

13. The omni-directional positioning unit of claim 11, wherein the base is a plate.

14. The omni-directional positioning unit of claim 13, wherein each said face is a plate.

15. The omni-directional positioning unit of claim 11, wherein the pyramid comprises a plurality of bars connecting corresponding vertices, respectively.

16. The omni-directional positioning unit of claim 11, wherein the base is configured as an equilateral triangle.

17. The omni-directional positioning unit of claim 16, wherein each said face is configured as an equilateral triangle or an arbitrary triangle.

18. The omni-directional positioning unit of claim 11, wherein the base is configured as an arbitrary triangle.

19. The omni-directional positioning unit of claim 18, wherein each said face is configured as an arbitrary triangle.

20. The omni-directional positioning unit of claim 11, wherein each said reflector is a reflective ball or an infrared reflector.
